(19) United States
(12) Patent Application Publication
(10) Pub. No.: US 2025/0186528 A1
(43) Pub. Date: Jun. 12, 2025

(54) ANTI-OBESITY AGENT AND ANTI-OBESITY COMPOSITION

(71) Applicant: MARUZEN PHARMACEUTICALS CO., LTD., Hiroshima (JP)

(72) Inventors: Mai KANAZAWA, Hiroshima (JP); Sawako IKEOKA, Hiroshima (JP); Mizuho TANABE, Hiroshima (JP)

(21) Appl. No.: 18/846,440

(22) PCT Filed: Feb. 21, 2023
(86) PCT No.: PCT/JP2023/006293

§ 371 Date: Sep. 12, 2024

(30) Foreign Application Priority Data
Mar. 15, 2022 (JP) 2022-040345

(51) Int. Cl. A61K 36/23; A61K 31/366; A61K 31/37; A61P 3/04

(52) U.S. Cl. CPC A61K 36/23; A61K 31/366; A61K 31/37; A61P 3/04

(57) ABSTRACT

An anti-obesity agent includes an extract of a below-ground portion of Peucedanum japonicum. An anti-obesity composition includes the anti-obesity agent.

### TECHNICAL FIELD

The present invention relates to anti-obesity agents and anti-obesity compositions.

### BACKGROUND ART

In recent years, body fat has increased due to lifestyle habits, such as satiation and lack of exercise, and obesity has become more common. Such increase in obesity is observed not only among humans, but also among pets or livestock. Obesity causes lifestyle-related diseases such as hyperlipidemia and arteriosclerosis. Therefore, the obesity is not only a cosmetic problem but also a serious health problem.

Cyclic AMP (CAMP) is known to be involved in lipolysis in vivo. The CAMP activates lipase present in the living body, and the fat is decomposed into fatty acid and glycerol by the activated lipase. However, activation of CAMP-phosphodiesterase induces degradation of CAMP and inhibits activation of lipase. Therefore, it is considered that the amount of CAMP in cells is increased by inhibiting the activity of CAMP-phosphodiesterase, and lipolysis can be facilitated.

Attempts have been made to extract substances having cyclic AMP phosphodiesterase inhibitory activity from natural products, and for example, Japanese tea extract (see, for example, Patent Document 1), Acer plant extract (see, for example, Patent Document 2), and the like have been reported.

However, there is still a strong demand for a novel material which has an anti-obesity effect and is highly safe to be widely usable as substances for food and drink products, cosmetic products, research reagents, and the like. Prompt development of such a novel material is currently desired.

### CITATION LIST

### Patent Documents

Patent Document 1: Japanese Unexamined Patent Application Publication No. 2003-012532
Patent Document 2: Japanese Unexamined Patent Application Publication No. 2003-113068

### SUMMARY OF INVENTION

### Technical Problem

The present invention aims to solve the above-described various problems existing in the related art, and achieve the following object. Specifically, the present invention has an object to provide an anti-obesity agent and an anti-obesity composition, which have an excellent anti-obesity effect and are highly safe.

### Solution to Problem

As a result of intensive studies to solve the above problems, the present inventors have found that an extract of a below-ground portion of Peucedanum japonicum has an excellent anti-obesity effect, is highly safe, and is effective for anti-obesity.

The present invention is based on the insights of the present inventors, and the means for solving the above problems are as follows.

<1> An anti-obesity agent including an extract of a below-ground portion of Peucedanum japonicum.

<2> The anti-obesity agent according to <1>, wherein the extract of the below-ground portion of Peucedanum japonicum is an extract obtained using a mixed solvent including water and a hydrophilic solvent.

<3> The anti-obesity agent according to <1> or <2>, wherein the extract of the below-ground portion of Peucedanum japonicum includes, as khellactone esters, hyuganin D, Peucedanocoumarin III, cis-3′-acetyl-4′-tigloylkhellactone, trans-3′-acetyl-4′-senecioylkhellactone, isosamidin, and pteryxin.

<4> The anti-obesity agent according to any one of <1> to <3>, wherein the anti-obesity agent has one or more functions selected from the group consisting of a function of inhibiting cyclic AMP phosphodiesterase activity, a function of facilitating differentiation induction of preadipocytes, a function of inhibiting lipopexia, and a function of inducing cell death of adipocytes.

<5> An anti-obesity composition including the anti-obesity agent according to any one of <1> to <4>.

### Effects of Invention

According to the anti-obesity agent and anti-obesity composition of the present invention, the above-described various problems existing in the related art can be solved, the above object can be achieved, and an anti-obesity agent and an anti-obesity composition, which have an excellent anti-obesity effect and are highly safe, can be provided.

### DESCRIPTION OF EMBODIMENTS

### (Anti-Obesity Agent)

The anti-obesity agent of the present invention includes, as an active ingredient, an extract of a below-ground portion of Peucedanum japonicum, and may further include other ingredients as necessary.

In the present specification, the term “obesity” means a state where a body fat is excessively accumulated. In the present specification, moreover, the term “anti-obesity” means reduction in excessively accumulated body fat to bring closer to a normal state. In addition, prevention of increase or accumulation of body fat to reduce body fat to bring to a normal state is also included in the context of the term “anti-obesity.” Note that, an amount of body fat does not need to be back to a normal state, as long as the amount of the body fat is reduced from the excessively accumulated state.

The details of substance(s) exhibiting an anti-obesity effect, which is included in the extract of the below-ground portion of Peucedanum japonicum, are not clearly understood yet. However, the fact that the extract of the below-ground portion of Peucedanum japonicum has an excellent anti-obesity effect and is effective as an anti-obesity agent has not been known at all, and is a novel insight attained by the present inventors.

<Extract of Below-Ground Portion of Peucedanum japonicum>

The Peucedanum japonicum (coastal hog fennel) (binomial name: Peucedanum japonicum Thunb.) is a plant assigned to the genus Peucedanum of the family Apiaceae, and is a perennial plant grown on the coast. The Peucedanum japonicum is distributed in the area extending toward the west from the Kanto region of Japan, in the Shikoku region, Kyushu region, and Okinawa region in Japan, in Korea, on the Chinese continent, in the Philippines, and the like, and are readily available from the above regions.

As the extract of the below-ground portion of Peucedanum japonicum, an extract prepared from an extraction portion used as an extraction raw material may be used, or a commercial product may be used.

The extraction portion of the Peucedanum japonicum is not particularly limited as long as the extraction portion is the below-ground portion of the Peucedanum japonicum, and may be appropriately selected according to the intended purpose. Examples of the extraction portion include roots, rhizomes, and the like. The above-listed examples may be used alone or in combination.

A shape, structure, and size of the extraction raw material of the Peucedanum japonicum are not particularly limited, and may be appropriately selected according to the intended purpose.

A preparation method of the extraction portion of the Peucedanum japonicum is not particularly limited, and may be appropriately selected according to the intended purpose. Examples of the preparation method include a method in which the extraction portion is dried, and the dried product is ground as it is or by a grinder, and the like. The dried product may be provided to solvent extraction as it is or after being ground. The drying may be performed in the sun or using a typically used dryer.

The extract of the below-ground portion of Peucedanum japonicum can be easily obtained by a method typically used for extraction of plants. An embodiment of the extract of the below-ground portion of Peucedanum japonicum is not particularly limited, and may be appropriately selected according to the intended purpose. Examples thereof include an extract liquid as it is, a diluted solution of the extract liquid, a concentrate of the extract liquid, dried products of the foregoing, crudely purified products of the foregoing, and purified products of the foregoing.

A method of the extraction is not particularly limited, and may be appropriately selected according to the intended purpose. Examples of the method include a method of extracting using any extraction device at room temperature or under reflux with heating, and the like. More specifically, the examples include a method in which the extraction portion of the Peucedanum japonicum that is an extraction raw material is added to a processing tank filled with an extraction solvent, while stirring as appropriate, the resultant mixture is left to stand for 30 minutes to 4 hours to elute a soluble component, followed by filtering to remove the extraction residue to obtain an extract liquid. The extract liquid may be further distilled to remove the extraction solvent and dried.

Moreover, the extraction raw material may be subjected to a pretreatment, such as degreasing with a nonpolar solvent (e.g., hexane, etc.) before use. By performing the pretreatment, such as degreasing, the extraction process with a polar solvent can be efficiently performed.

Conditions (extraction time and extraction temperature) for the extraction of the Peucedanum japonicum, the extraction solvent, and a usage amount of the extraction solvent is not particularly limited, and may be appropriately selected according to the intended purpose.

The extraction solvent is not particularly limited, and may be appropriately selected according to the intended purpose. Examples of the extraction solvent include water, hydrophilic solvents, mixed solvents including water and a hydrophilic solvent, and the like. Among the above-listed examples, a mixed solvent including water and a hydrophilic solvent is preferred in view of an excellent anti-obesity effect of the extract.

The water is not particularly limited, and may be appropriately selected according to the intended purpose. Examples of the water include pure water, tap water, well water, spring water, mineral water, hot spring water, spring water, fresh water, and the foregoing water subjected to various processes. Examples of the processes performed on the water include purification, heating, sterilization, filtration, ion exchange, adjustment of osmotic pressure, buffering, and the like. Note that, the water that can be used for the extraction solvent includes purified water, hot water, ion-exchanged water, physiological saline, phosphate buffers, phosphate buffered saline, and the like. The above water may be used alone or in combination.

The hydrophilic solvent is not particularly limited, and may be appropriately selected according to the intended purpose. Examples of the hydrophilic solvent include: C1-C5 lower alcohols, such as methanol, ethanol, propyl alcohol, isopropyl alcohol, etc.; lower aliphatic ketones, such as acetone, methyl ethyl ketone, etc.; C2-C5 polyhydric alcohols, such as 1,3-butylene glycol, propylene glycol, glycerin, etc.; and the like. The above-listed examples may be used alone or in combination.

An amount of the hydrophilic solvent used in the mixed solvent relative to the water is not particularly limited, and may be appropriately selected according to the intended purpose. In the case where a lower alcohol is used as the hydrophilic solvent, the amount of lower alcohol added is preferably 1 part by volume to 90 parts by volume relative to 10 parts by volume of water. In the case where a lower aliphatic ketone is used as the hydrophilic solvent, the amount of the lower aliphatic ketone added is preferably 1 part by volume to 40 parts by volume relative to 10 parts by volume of water. In the case where a polyhydric alcohol is used as the hydrophilic solvent, the amount of the polyhydric alcohol added is preferably 1 part by volume to 90 parts by volume relative to 10 parts by volume of water.

A temperature of the extraction solvent is not particularly limited, and may be appropriately selected according to the intended purpose. The extraction solvent is preferably used at room temperature or higher and a boiling point of the solvent or lower.

The obtained extract of the below-ground portion of Peucedanum japonicum may be subjected to a process, such as dilution, concentration, drying, purification, and the like according to common practice in order to obtain a diluted product, condensate, dried product, crudely purified product, purified product, or the like of the extraction of the below-ground portion of Peucedanum japonicum.

A purification method of the extraction of the below-ground portion of Peucedanum japonicum is not particularly limited, and may be appropriately selected according to the intended purpose. Examples of the purification method include purification methods, such as processing with activated carbon, processing with adsorption resins, processing with ion-exchange resins, and the like. Purification of the extract by the above purification method can increase a concentration of an active ingredient and remove unwanted substances.

The obtained extract of the below-ground portion of Peucedanum japonicum can be used as it is as the anti-obesity agent, but the concentrate of the extract and the dried product of the extract are preferably used in view of easiness of use. When the dried product is obtained, a carrier, such as dextrin or cyclodextrin may be added to improve hygroscopicity.

From the viewpoint of achieving an excellent anti-obesity effect, a preferable embodiment of the extract of the below-ground portion of Peucedanum japonicum includes, as khellactone esters, hyuganin D, Peucedanocoumarin III, cis-3′-acetyl-4′-tigloylkhellactone, trans-3′-acetyl-4′-senecioylkhellactone, isosamidin, and pteryxin.

A total amount of the khellactone esters in the extract of the below-ground portion of Peucedanum japonicum is not particularly limited, and may be appropriately selected according to the intended purpose. The total amount of the khellactone esters is preferably from 1% by mass to 5% by mass, and more preferably from 2.5% by mass to 5% by mass.

An amount of the hyuganin D in the extract of the below-ground portion of Peucedanum japonicum is not particularly limited, and may be appropriately selected according to the intended purpose. For example, the amount of the hyuganin D is from 0.04% by mass to 0.5% by mass.

An amount of the Peucedanocoumarin III in the extract of the below-ground portion of Peucedanum japonicum is not particularly limited, and may be appropriately selected according to the intended purpose. For example, the amount of the Peucedanocoumarin III is from 0.1% by mass to 1.0% by mass.

Amount of the cis-3′-acetyl-4′-tigloylkhellactone in the extract of the below-ground portion of Peucedanum japonicum is not particularly limited, and may be appropriately selected according to the intended purpose. For example, the amount of the cis-3′-acetyl-4′-tigloylkhellactone is from 0.01% by mass to 0.5% by mass.

An amount of the trans-3′-acetyl-4′-senecioylkhellactone in the extract of the below-ground portion of Peucedanum japonicum is not particularly limited, and may be appropriately selected according to the intended purpose. The amount of the trans-3′-acetyl-4′-senecioylkhellactone is preferably from 0.05% by mass to 2.5% by mass, and more preferably from 0.3% by mass to 2.0% by mass.

An amount of the isosamidin in the extract of the below-ground portion of Peucedanum japonicum is not particularly limited, and may be appropriately selected according to the intended purpose. The amount of the isosamidin is preferably from 0.01% by mass to 0.5% by mass, and more preferably from 0.05% by mass to 0.4% by mass.

An amount of the pteryxin in the extract of the below-ground portion of Peucedanum japonicum is not particularly limited, and may be appropriately selected according to the intended purpose. For example, the amount of the pteryxin is from 0.5% by mass to 5% by mass.

A measuring method of an amount of the khellactone esters in the extraction of the below-ground portion of Peucedanum japonicum is not particularly limited, and may be appropriately selected according to the intended purpose. Examples of the measuring method include the method described in below-described Test Example 1-1, and the like.

The extract of the below-ground portion of Peucedanum japonicum may include chlorogenic acid and rutin.

An amount of the chlorogenic acid in the extract of the below-ground portion of Peucedanum japonicum is not particularly limited, and may be appropriately selected according to the intended purpose. The amount of the chlorogenic acid is preferably 1.0% by mass or less, and more preferably from 0.05% by mass to 0.8% by mass.

An amount of the rutin in the extract of the below-ground portion of Peucedanum japonicum is not particularly limited, and may be appropriately selected according to the intended purpose. The amount of the rutin is preferably 0.1% by mass or less, and more preferably from 0.001% by mass to 0.05% by mass.

A measuring method of the amount of the chlorogenic acid and the amount of the rutin in the extract of the below-ground portion of Peucedanum japonicum is not particularly limited, and may be appropriately selected according to the intended purpose. Examples of the measuring method include the method described in below-described Test Example 1-2, and the like.

An amount of the extract of the below-ground portion of Peucedanum japonicum in the anti-obesity agent is not particularly limited, and may be appropriately adjusted according to pharmacological activity of the extract of the below-ground portion of Peucedanum japonicum or the like. The anti-obesity agent may be composed of the extract of the below-ground portion of Peucedanum japonicum alone.

### <Other Ingredients>

The above other ingredients are not particularly limited, and may be appropriately selected according to a usage form of the anti-obesity agent. Examples of the above other ingredients include excipients, moisture-proof agents, preservatives, enhancers, thickeners, emulsifiers, antioxidants, sweeteners, acidulants, seasonings, coloring agents, fragrances, whitening agents, humectants, oil components, UV absorbers, surfactants, thickeners, alcohols, powder components, colorants, aqueous components, water, skin nutrients, and the like. The above-listed examples may be used alone or in combination.

An amount of the above other ingredients in the anti-obesity agent is not particularly limited, and may be appropriately selected according to the intended purpose.

### Use

Use of the anti-obesity agent is not particularly limited, and may be appropriately selected according to the intended purpose. Examples of use include pharmaceutical products, quasi-drugs, food and drink products, cosmetic products, and the like.

Since the anti-obesity agent has an excellent anti-obesity effect and is highly safe, the anti-obesity agent is suitably used, for example, as an active ingredient of an anti-obesity composition.

The anti-obesity agent is suitably applied to humans, but can be apply to animals other than humans (e.g., mice, rats, hamsters, dogs, cats, cows, pigs, monkeys, etc.) as long as the functions and effects of the anti-obesity agent can be exhibited.

Usage of the anti-obesity agent is not particularly limited, and may be appropriately selected according to the intended purpose. Examples of the usage include oral administration, parenteral administration, external application, and the like.

A dosage form of the anti-obesity agent is not particularly limited, and may be appropriately selected according to the intended purpose. Examples of the dosage form include: oral preparations, such as tablets, powders, capsules, granules, extracts, syrups, and the like; parenteral preparations, such as injections, drips, suppositories, and the like; external preparations, such as toners, emulsified lotions, cream, ointments, serums, lotions, face masks, gel, lip balm, lip sticks, foundation, bath salts, soaps, body wash, astringents, hair tonics, hair lotions, hair cream, hair liquids, pomades, shampoos, conditioners, hair masks, and the like; etc.

A production method of each of the above dosage forms of the anti-obesity agent is not particularly limited and may be appropriately selected according to any method known in the related art.

A usage method of the anti-obesity agent, such as a usage amount, usage duration, or the like of the anti-obesity agent, is not particularly limited, and may be appropriately selected according to the intended purpose.

Moreover, the anti-obesity agent of the present invention can be also used as a reagent for research about an activity mechanism of the anti-obesity effect.

The anti-obesity effect of the extraction of the below-ground portion of Peucedanum japonicum can be achieved by one or more functions selected from the group consisting of a function of inhibiting cyclic AMP phosphodiesterase activity, a function of facilitating differentiation induction of preadipocytes, a function of inhibiting lipopexia, and a function of inducing cell death of adipocytes. Therefore, the anti-obesity agent preferably has one or more functions selected from the group consisting of a function of inhibiting cyclic AMP phosphodiesterase activity, a function of facilitating differentiation induction of preadipocytes, a function of inhibiting lipopexia, and a function of inducing cell death of adipocytes. Note that, the anti-obesity effect of the extraction of the below-ground portion of Peucedanum japonicum is not limited to one or more functions selected from the group consisting of a function of inhibiting cyclic AMP phosphodiesterase activity, a function of facilitating differentiation induction of preadipocytes, a function of inhibiting lipopexia, and a function of inducing cell death of adipocytes.

Moreover, the present invention also relates to a cyclic AMP phosphodiesterase activity inhibitor, preadipocyte differentiation induction promoter, lipopexia inhibitor, or an adipocyte cell death inducer, which includes the extract of the below-ground portion of Peucedanum japonicum.

### (Anti-Obesity Composition)

The anti-obesity composition of the present invention includes the anti-obesity agent of the present invention, and may further include other ingredients as necessary.

### <Anti-Obesity Agent>

The anti-obesity agent is the above-described anti-obesity agent of the present invention.

An amount of the anti-obesity agent in the anti-obesity composition is not particularly limited, and can be appropriately adjusted according to a usage form of the anti-obesity composition, a pharmacological activity of the Peucedanum japonicum extract, or the like. The amount of the anti-obesity agent that is converted as an amount of the Peucedanum japonicum extract is preferably from 0.0001% by mass to 20% by mass, and more preferably from 0.0001% by mass to 10% by mass. The anti-obesity composition may be composed of the anti-obesity agent alone.

### <Other Ingredients>

Other ingredients in the anti-obesity composition are not particularly limited, and may be appropriately selected according to a usage form of the anti-obesity composition. Examples of the above other ingredients include the same ingredients as the other ingredients described in the above section of the anti-obesity agent. Such ingredients may be used alone or in combination.

An amount of the above other ingredients in the anti-obesity composition is not particularly limited, and may be appropriately selected according to the intended purpose.

### Embodiments

Embodiments of the anti-obesity composition are not particularly limited, and may be appropriately selected according to the intended purpose. Examples of the embodiments include pharmaceutical products, quasi-drugs, drink and food products, cosmetic products, and the like.

The anti-obesity composition of the present invention can be used daily, and can extremely effectively exhibit a variety of pharmacological activity including an anti-obesity effect due to the action of the extract of the below-ground portion of Peucedanum japonicum serving as an active ingredient.

The anti-obesity composition is suitably applied to humans, but can be apply to animals other than humans (e.g., mice, rats, hamsters, dogs, cats, cows, pigs, monkeys, etc.) as long as the functions and effects of the anti-obesity composition can be exhibited.

Usage of the anti-obesity composition of the present invention is not particularly limited, and may be appropriately selected according to the intended purpose. Examples of the usage include oral administration, parenteral administration, external application, and the like.

Examples of the composition for the oral administration include the above-described oral preparations, food and drink products, and the like. In the present specification, the food and drink products refer to products that have little risk for harming human health and can be consumed in ordinary social life through oral administration or gastrointestinal administration. The food and drink products are not limited to categories according to the administrative classification, such as foods, medical products, and quasi-drugs. Accordingly, the food and drink products include a wide range of food and drink products including general food products, health foods (functional food and drink products), health-promoting food products (food for specified health uses, food with nutrient function claims, and food with functional claims), quasi-drugs, pharmaceutical products, etc., which are for oral intake.

A type of the composition for the oral administration is not particularly limited, and may be appropriately selected according to the intended purpose. Examples of the composition for the oral administration include: beverages such as tea beverages, soft drinks, carbonated beverages, nutritional beverages, fruit-based beverages, lactic beverages, alcoholic beverages, coffee beverages, coffee-containing soft drinks, and the like (including concentrates of the foregoing beverages and powders for preparing the foregoing beverages); frozen desserts such as ice creams, sherbets, shaved ice, and the like; noodles, such as buckwheat noodles, udon noodles, starch noodles, Gyoza skins, Shumai skins, Chinese noodles, instant noodles, and the like; confectionary, such as hard candies, candies, chewing gums, chocolate, tablets, snacks, biscuits, jelly, jams, creams, sweet pastries, bread, and the like; fishery products, such as crabs, salmon, clams, tuna, sardine, shrimps, bonito, mackerel, whale, oysters, Pacific saury, squid, Anadora broughtonii, scallops, abalone, sea urchin, salmon roe, Sulculus diversicolor supertexta, and the like; fish or meat processed foods, such as fish paste, ham, sausages, and the like; dairy products, such as processed milk, fermented milk, and the like; fat or oil, or fat or oil processed food products, such as salad oil, tempura oil, margarines, mayonnaise, shortening, whipping creams, dressing, and the like; seasonings, such as sources and dip sources; retort pouch-packaged foods, such as curry, stew, Oyakodon, gruel, rice soups, Chukadon, a bowl of rice with breaded pork cutlets, a bowl of rice with tempura, Unadon, Hayashi rice, Oden, Mapo tofu, a bowl of rice with beef, Bolognese sauces, egg soup, omelet rice, Gyoza, Shumai, hamburgers, meat balls, and the like; daily dishes, such as salads and pickles; various forms of health, cosmetic, or nutritional supplementary foods; pharmaceutical products and quasi-drugs, such as tablets, granules, capsules, drink preparations, lozenge, mouthwash, and the like; oral freshener used inside the mouth, such as mouth freshener and breath freshener; tooth paste; and the like.

Examples of the compositions for other than the oral administration include the above-described parenteral preparations and external preparations. For example, ointments, creams, emulsified lotions, locations, masks, foundations, and the like can be used as skin cosmetics, and hair tonics, hair creams, hair liquids, shampoos, pomades, conditioners, and the like can be used as hair cosmetics.

A production method of the anti-obesity composition is not particularly limited, and may be appropriately selected according to a usage form of the anti-obesity composition.

An amount of use, duration of use, and the like of the anti-obesity composition are not particularly limited, and may be appropriately selected according to the intended purpose.

As described above, the anti-obesity agent and the anti-obesity composition of the present invention have an excellent anti-obesity effect.

Accordingly, the present invention is also related to an anti-obesity method in which the anti-obesity agent, the anti-obesity composition, or both are administered to a subject.

### EXAMPLES

Production Examples, Test Examples, and Formulation Examples of the present invention will be described hereinafter, but the present invention is not limited to Production Examples, Test Examples, and Formulation Examples in any way.

Test samples used in Test Examples 1 to 4 described below were produced in the following manner.

### Production Example 1: 30% Ethanol Extract of Below-Ground Portion of Peucedanum japonicum

An extract of the below-ground portion of Peucedanum japonicum with 30% by volume ethanol was produced in the following manner.

To 10 g of the below-ground portion of Peucedanum japonicum, 200 mL of 30% by volume ethanol was added, and extraction was performed by a reflux condenser at 80° C. for 1 hour, followed by filtering the resultant solution with filter paper to obtain an extract liquid.

The obtained extract liquid was concentrated under reduced pressure, and dried, to thereby yield 2.28 g of a 30% by volume ethanol extract (powder) of the below-ground portion of Peucedanum japonicum.

### Production Example 2: 50% Ethanol Extract of Below-Ground Portion of Peucedanum japonicum

An extract of the below-ground portion of Peucedanum japonicum with 50% by volume ethanol was produced in the following manner.

A 50% by volume ethanol extract (powder) of Peucedanum japonicum in an amount of 2.23 g was obtained in the same manner as in Production Example 1, except that the 30% by volume ethanol was replaced with 50% by volume ethanol.

### Production Example 3: 80% Ethanol Extract of Below-Ground Portion of Peucedanum japonicum

An extract of the below-ground portion of Peucedanum japonicum with 80% by volume ethanol was produced in the following manner.

A 80% by volume ethanol extract (powder) of Peucedanum japonicum in an amount of 1.90 g was obtained in the same manner as in Production Example 1, except that the 30% by volume ethanol was replaced with 80% by volume ethanol.

### Comparative Production Example 1: 30% Ethanol Extract of Above-Ground Portion of Peucedanum japonicum

An extract of the above-ground portion of Peucedanum japonicum with 30% by volume ethanol was produced in the following manner.

A 30% by volume ethanol extract (powder) of the above-ground portion of Peucedanum japonicum in an amount of 10.48 g was obtained in the same manner as in Production Example 1, except that 10 g of the below-ground portion of Peucedanum japonicum was replaced with 30 g of the above-ground portion of Peucedanum japonicum, and 200 mL of the 30% by volume ethanol was replaced with 600 mL of 30% by volume ethanol.

### Comparative Production Example 2: 50% Ethanol Extract of Above-Ground Portion of Peucedanum japonicum

An extract of the above-ground portion of Peucedanum japonicum with 50% by volume ethanol was produced in the following manner.

A 50% by volume ethanol extract (powder) of the above-ground portion of Peucedanum japonicum in an amount of 10.28 g was obtained in the same manner as in Comparative Production Example 1, except that the 30% by volume ethanol was replaced with 50% by volume ethanol.

### Comparative Production Example 3: 80% Ethanol Extract of Above-Ground Portion of Peucedanum japonicum

An extract of the above-ground portion of Peucedanum japonicum with 80% by volume ethanol was produced in the following manner.

A 80% by volume ethanol extract (powder) of the above-ground portion of Peucedanum japonicum in an amount of 9.27 g was obtained in the same manner as in Comparative Production Example 1, except that the 30% by volume ethanol was replaced with 80% by volume ethanol.

### Test Example 1-1: Measurement of Khellactone Esters Content

A khellactone esters content in each of the Peucedanum japonicum extracts produced in Production Examples 1 to 3 and Comparative Production Examples 1 to 3 was measured in the following manner.

### <Preparation of Analysis Sample>

Each sample was precisely weighed by 10 mg in a 10 mL volumetric flask and a volume of the content of the volumetric flask was adjusted to 10 mL with 50% acetonitrile (ACN). The resultant liquid was filtered with a 0.45 μm hydrophilic polytetrafluoroethylene (PTFE) membrane filter to obtain a sample solution.

As a standard, (+)-pteryxin (ChemFaces, Lot. CFS201901, purity of 98% or greater) was used, and the amount of khellactone esters (hyuganin D, Peucedanocoumarin III, cis-3′-acetyl-4′-tigloylkhellactone, trans-3′-acetyl-4′-senecioylkhellactone, Peucedanocoumarin II, isosamidin, and pteryxin) was determined as purity of 100%. The weighed amount was approximately 2 mg.

### <HPLC Analysis Conditions>

Column: Wakosil II 5C18AR, 4.6 mm×250 mm (produced by FUJI FILM Wako Pure Chemical Corporation)
Mobile phase: 0.1% TFA (A): ACN (B), 0 min to 45 min (41% B)
Flow rate: 1.0 mL/min
Oven temperature: 40° C.
Injection amount: 20 μL
Detection: 320 nm

The khellactone esters content (pteryxin-converted value) in each of the Peucedanum japonicum extracts produced in Production Examples 1 to 3 and Comparative Production Examples 1 to 3 is presented in Table 1-1 below.

TABLE 1-1

| | Extraction | Extraction | Amount % | | | | | | | |
|---|---|---|---|---|---|---|---|---|---|---|
| | portion | solvent | A | B | C | D | E | F | G | Total |
| Produc- | Below- | 30% by | 0.11 | 0.47 | 0.12 | 0.78 | 0.022 | 0.17 | 1.20 | 2.87 |
| tion | ground | volume | | | | | | | | |
| Example | portion | ethanol | | | | | | | | |
| 1 | | | | | | | | | | |
| Produc- | Below- | 50% by | 0.13 | 0.54 | 0.13 | 0.97 | 0.027 | 0.19 | 1.34 | 3.33 |
| tion | ground | volume | | | | | | | | |
| Example | portion | ethanol | | | | | | | | |
| 2 | | | | | | | | | | |
| Produc- | Below- | 80% by | 0.12 | 0.51 | 0.12 | 0.86 | 0.024 | 0.16 | 1.28 | 3.07 |
| tion | ground | volume | | | | | | | | |
| Example | portion | ethanol | | | | | | | | |
| 3 | | | | | | | | | | |
| Compara- | Above- | 30% by | 0.16 | 0.61 | 0.087 | 0.17 | 0.053 | 0.021 | 0.85 | 1.95 |
| tive | ground | volume | | | | | | | | |
| Produc- | portion | ethanol | | | | | | | | |
| tion | | | | | | | | | | |
| Example | | | | | | | | | | |
| 1 | | | | | | | | | | |
| Compara~ | Above- | 50% by | 0.18 | 0.68 | 0.098 | 0.19 | 0.057 | 0.013 | 0.94 | 2.16 |
| tive | ground | volume | | | | | | | | |
| Produc- | portion | ethanol | | | | | | | | |
| tion | | | | | | | | | | |
| Example | | | | | | | | | | |
| 2 | | | | | | | | | | |
| Compara- | Above- | 80% by | 0.22 | 0.82 | 0.12 | 0.21 | 0.068 | 0.015 | 1.16 | 2.39 |
| tive | ground | volume | | | | | | | | |
| Produc- | portion | ethanol | | | | | | | | |
| tion | | | | | | | | | | |
| Example | | | | | | | | | | |

A: hyuganin D
B: Peucedanocoumarin III
C: cis-3′-acetyl-4′-tigloylkhellactone
D: trans-3′-acetyl-4′-senecioylkhellactone
E: Peucedanocoumarin II
F: isosamidin
G: pteryxin

### Test Example 1-2: Measurement of Chlorogenic Acid Content and Rutin Content

A chlorogenic acid content and a rutin content in each of the Peucedanum japonicum extracts produced in Production Examples 1 to 3 and Comparative Production Examples 1 to 3 were measured in the following manner.

### <Preparation of Analysis Sample>

Each sample was precisely weighed by 10 mg in a 10 mL volumetric flask and a volume of the content of the volumetric flask was adjusted to 10 mL with 20% methanol. The resultant liquid was filtered with a 0.45 μm hydrophilic polytetrafluoroethylene (PTFE) membrane filter to obtain a sample solution.

As standards, chlorogenic acid (MP Bio Japan K.K.) and rutin (FUJI FILM Wako Pure Chemical Corporation) were used, and the amounts of chlorogenic acid and rutin in the sample solution were determined as purity of 100%. The weighed amount was approximately 5 mg.

### <HPLC Analysis Conditions>

Column: Wakosil II 5C18AR, 4.6 mm×250 mm (produced by FUJI FILM Wako Pure Chemical Corporation)
Mobile phase: 0.1% TFA (A):ACN (B)
   0 min to 25 min (7%-30% B),
   25.01 min to 30 min (90% B)
   30.01 min to 40 min (7% B)
Flow rate: 1.0 mL/min
Oven temperature: 30° C.
Injection amount: 5 μL, 10 μL, 20 μL
Detection: 330 nm (chlorogenic acid), 350 nm (rutin)

The chlorogenic acid content and the rutin content in each of the Peucedanum japonicum extracts produced in Production Examples 1 to 3 and Comparative Production Examples 1 to 3 are presented in Table 1-2 below.

TABLE 1-2

| | | | Amount % | |
|---|---|---|---|---|
| | Extraction | Extraction | Chlorogenic | |
| | portion | solvent | acid | Rutin |
| Production | Below- | 30% by | 0.11 | 0.0027 |
| Example 1 | ground | volume | | |
| | portion | ethanol | | |
| Production | Below- | 50% by | 0.11 | 0.0029 |
| Example 2 | ground | volume | | |
| | portion | ethanol | | |
| Production | Below- | 80% by | 0.14 | 0.011 |
| Example 3 | ground | volume | | |
| | portion | ethanol | | |
| Comparative | Above- | 30% by | 1.64 | 0.31 |
| Production | ground | volume | | |
| Example 1 | portion | ethanol | | |
| Comparative | Above- | 50% by | 1.81 | 0.33 |
| Production | ground | volume | | |
| Example 2 | portion | ethanol | | |
| Comparative | Above- | 80% by | 2.04 | 0.40 |
| Production | ground | volume | | |
| Example 3 | portion | ethanol | | |

### Test Example 2: Test for Function of Inhibiting Cyclic AMP Phosphodiesterase Activity

Each of the Peucedanum japonicum extracts produced in Production Examples 1 to 3 and Comparative Production Examples 1 to 3 was used as a test sample, and a function of inhibiting cyclic AMP phosphodiesterase activity was tested by the following test method.

To 0.2 mL of a 50 mmol/L Tris-HCl buffer solution (pH 7.5) including 5 mmol/L magnesium chloride, 0.1 mL of a 2.5 mg/mL bovine serum albumin solution, 0.1 mL of a 0.1 mg/mL cyclic AMP phosphodiesterase solution, and 0.05 mL of the test sample solution (refer to Table 2 below for the sample concentration), and the resultant mixture was left to stand for 5 minutes at 37° C. to carry out a preliminary reaction. To the resultant reaction solution, 0.05 mL of a 0.5 mg/mL cyclic AMP (CAMP) solution was added and the resultant mixture was allowed to react for 60 minutes at 37° C. Thereafter, the reaction solution was boiled over a boiling water bath for 3 minutes to stop the reaction. The resultant reaction solution was centrifuged (for 10 minutes at 2,260×g, and at 4° C.). The cyclic AMP as a reaction substrate in the supernatant was analyzed through high performance liquid chromatography (HPLC) under the following conditions. Moreover, the same procedure was performed by adding only a solvent without the sample, as a control.

### <HPLC Conditions>

Pump: DP-8020 (Tosoh Corporation)
UV-visible detector: UV-8020 (Tosoh Corporation)
Integrator: Chromatocorder 21 (Tosoh Corporation)
Column oven: CO-8020 (Tosoh Corporation)
Online degasser: SD-8022 (Tosoh Corporation)
Column: Wakosil C18-ODS 5 μm (FUJI FILM Wako Pure Chemical Corporation)
Mobile phase: 1 mmol/L TBAP in 25 mmol/L KH₂PO₄:CH₃CN=90:10
Flow rate: 1.0 mL/min
Detection: 260 nm
Atten: 128

Next, a peak area (A) of the cyclic AMP standard, a peak area (B1) of the supernatant of the reaction solution between the cyclic AMP standard to which the test sample was not added and cyclic AMP phosphodiesterase, and a peak area (B2) of the supernatant of the reaction solution between the cyclic AMP standard to which the test sample was added and cyclic AMP phosphodiesterase were determined. From the results obtained, a degradation rate (C) of the cyclic AMP standard without the test sample and a degradation rate (D) of the cyclic AMP standard with the test sample were calculated by the following equations. Degradation rate (C) (%) of cyclic AMP sample without test

$$sample = ( 1 - B 1 / A ) \times 100$$

$$Degradation rate ( D ) ( \% ) of cyclic AMP with test sample = ( 1 - B 2 / A ) \times 100$$

Then, based on the degradation rates (C and D) calculated by the above equations, a cyclic AMP phosphodiesterase activity inhibition rate (%) was calculated by the following equation. The results are presented in Table 2.

$$Cyclic AMP phosphodiesterase activity inhibition rate ( \% ) = ( 1 - D / C ) \times 100$$

TABLE 2

| | | | | cAMP |
|---|---|---|---|---|
| Test Sample | | | | phosphodiesterase |
| | | | Concentration | activity inhibition |
| Type | | | (μg/mL) | rate (%) |
| Peucedanum | Below- | 30% ethanol | 50 | 3.5 |
| japonicum | ground | extract | | |
| | portion | 50% ethanol | 50 | 7.8 |
| | | extract | | |
| | | 80% ethanol | 50 | 2.3 |
| | | extract | 200 | 27.8 |
| | Above- | 30% ethanol | 50 | −2.3 |
| | ground | extract | | |
| | portion | 50% ethanol | 50 | 4.2 |
| | | extract | | |
| | | 80% ethanol | 50 | −7.4 |
| | | extract | 200 | 7.8 |

It was confirmed from the results of Table 2 that the extracts of the below-ground portion of Peucedanum japonicum had an excellent function of inhibiting cyclic AMP phosphodiesterase activity.

### Test Example 3: Test for Function of Facilitating Differentiation Induction Using Human Visceral Adipocytes

Each of the Peucedanum japonicum extracts produced in Production Examples 1 to 6 was used as a test sample, and a function of facilitating differentiation induction was tested by the following test method.

Human visceral preadipocytes were precultured in a growth medium (PGM BulletKit (Takara Bio Inc.)) in a 75 cm² flask at 37° C. in 5% CO₂, and the cells were collected by trypsinization.

The cells were dispensed into wells of a collagen-coated 96-well plate (IWAKI) using PGM BulletKit so that 2.5×10³ cells/200 μL were provided per well, and were cultured at 37° C. in 5% CO₂ until confluent. After the culture, the culture solution was discarded, and replaced with a differentiation induction medium. Simultaneously, a medium containing the predetermined concentration of the test sample was added, followed by culturing for 7 days at 37° C. in 5% CO₂. The culture supernatant was discarded, and the cells were further cultured for 8 days using PGM BulletKit.

After completing the culture, the culture solution was discarded, and intracellular triglyceride (TG) was measured using AdipoRed™ Assay Reagent (LONZA, USA). Thereafter, the protein was extracted by M-PER Mammalian Protein Extraction Reagent (PIERCE Biotechnology, Inc.), and the protein was quantified using BCA Protein Assay Reagent Kit (PIERCE Biotechnology, Inc.).

The results are presented in Table 3. Each numerical value (%) in Table 3 is a relative value when an amount of differentiated cells without the test sample is determined as 100%.

TABLE 3

| | | | | TG amount |
|---|---|---|---|---|
| | | | | relative to |
| Test Sample | | | | protein |
| | | | Concentration | amount |
| Type | | | (μg/mL) | (%) |
| Peucedanum | Below- | 30% ethanol | 50 | 112.4 |
| japonicum | ground | extract | 100 | 122.7 |
| | portion | | 400 | 227.4 |
| | | | 800 | 622.3 |
| | | 50% ethanol | 100 | 152.8 |
| | | extract | 400 | 378.1 |
| | | | 800 | 731.8 |
| | | 80% ethanol | 50 | 99.8 |
| | | extract | 100 | 131.3 |
| | | | 400 | 306.0 |
| | | | 800 | 747.9 |
| | Above- | 30% ethanol | 50 | 88.0 |
| | ground | extract | 100 | 66.7 |
| | portion | | 400 | 164.4 |
| | | | 800 | 218.4 |
| | | 50% ethanol | 100 | 90.2 |
| | | extract | 400 | 107.2 |
| | | | 800 | 182.3 |
| | | 80% ethanol | 50 | 98.8 |
| | | extract | 100 | 72.3 |
| | | | 400 | 88.4 |
| | | | 800 | 122.8 |

As presented in Table 3, the amount of the TG relative to the amount of the protein increased more when the extract of the below-ground portion of Peucedanum japonicum was added, and therefore it was confirmed that the extract of the below-ground portion of Peucedanum japonicum had an excellent function of facilitating differentiation induction of human visceral preadipocytes.

### Test Example 4: Test of Lipopexia Inhibition Function and Cell Death Induction Function Using Human Visceral Adipocytes

Each of the Peucedanum japonicum extracts produced in Production Examples 1 to 3 and Comparative Production Examples 1 to 3 was used as a test sample, and a lipopexia inhibition function and a cell death induction function were tested by the following test method.

Human visceral preadipocytes were precultured in a growth medium (PGM BulletKit (Takara Bio Inc.)) in a 75 cm² flask at 37° C. in 5% CO₂, and the cells were collected by trypsinization.

The cells were dispensed into wells of a collagen-coated 96-well plate (IWAKI) using PGM BulletKit so that 2.5×10³ cells/200 μL were provided per well, and were cultured at 37° C. in 5% CO₂ until confluent. After the culture, the culture solution was discarded, and replaced with a differentiation induction medium, followed by culturing for 7 days at 37° C. in 5% CO₂. The culture supernatant was discarded and replaced with PGM BulletKit. Simultaneously, a medium containing the predetermined concentration of the test sample was added, and the cells were further cultured for 8 days.

After completing the culture, the culture solution was discarded, and intracellular triglyceride (TG) was measured using AdipoRed™ Assay Reagent (LONZA, USA). Thereafter, the protein was extracted by M-PER Mammalian Protein Extraction Reagent (PIERCE Biotechnology, Inc.), and the protein was quantified using BCA Protein Assay Reagent Kit (PIERCE Biotechnology, Inc.).

The results are presented in Table 4. Each numerical value (%) in Table 4 is a relative value when an amount of differentiated cells without the test sample is determined as 100%.

TABLE 4

| | | | | | | TG amount |
|---|---|---|---|---|---|---|
| | | | | | | relative to |
| Test Sample | | | | TG | Protein | protein |
| | | | Concentration | amount | amount | amount |
| Type | | | (μg/mL) | (%) | (%) | (%) |
| Peucedanum | Below-ground | 30% ethanol | 800 | 64.1 | 81.7 | 78.6 |
| japonicum | portion | extract | | | | |
| | | 50% ethanol | 800 | 36.9 | 73.4 | 50.3 |
| | | extract | | | | |
| | Above-ground | 30% ethanol | 800 | 82.7 | 92.2 | 89.7 |
| | portion | extract | | | | |
| | | 50% ethanol | 800 | 71.4 | 90.3 | 79.1 |
| | | extract | | | | |

As presented in Table 4, the amount of the TG relative to the amount of the protein decreased more when the extract of the below-ground portion of Peucedanum japonicum was added, and therefore it was confirmed that the extract of the below-ground portion of Peucedanum japonicum had an excellent lipopexia inhibition function. Moreover, the amount of the protein also decreased when the extract of the below-ground portion of Peucedanum japonicum was added, and therefore it was confirmed that the extract of the below-ground portion of Peucedanum japonicum had an excellent cell death function.

### Production Example 4: 50% Ethanol Extract of Below-Ground Portion of Peucedanum japonicum

A 50% by volume ethanol extract (powder) of the above-ground portion of Peucedanum japonicum in an amount of 1.70 g was obtained in the same manner as in Production Example 2.

The extract obtained in Production Example 4 was subjected to the measurement of the khellactone esters content in the same manner as in Test Example 1-1 above. The results are as follows.

### <Khellactone Esters Content>

Hyuganin D: 0.13% by mass
Peucedanocoumarin III: 0.33% by mass
Cis-3′-acetyl-4′-tigloylkhellactone: 0.30% by mass
Trans-3′-acetyl-4′-senecioylkhellactone: 0.28% by mass
Peucedanocoumarin II: 0.015% by mass
Isosamidin: 0.049% by mass
Pteryxin: 1.16% by mass
Total: 2.26% by mass

A chlorogenic acid content and a rutin content in the extract obtained in Production Example 4 were measured in the same manner as in above-described Test Example 1-2. The results are as follows.
Chlorogenic acid: 0.80% by mass
Rutin: 0.015% by mass

The above functions of the extract obtained in Production Example 4 were tested in the same manner as in Test Examples 2 to 4. As a result, the same functions as with the extract of Production Example 2 were confirmed.

### Formulation Example 1

Tablets having the following formulation were prepared according to common practice.
Peucedanum japonicum extract of Production Example 1: 5.0 mg
Dolomite: 83.4 mg (including 20% of calcium and 10% of magnesium)
Casein phosphopeptide: 16.7 mg
Vitamin C: 33.4 mg
Maltitol: 136.8 mg
Collagen: 12.7 mg
Sucrose fatty acid ester: 12.0 mg

### Formulation Example 2

An oral liquid preparation having the following formulation was prepared according to common practice.

### <Formulation for One Ampule (100 mL Per Ampule)>

Peucedanum japonicum extract of Production Example 2: 0.3% by mass
Sorbit: 12.0% by mass
Sodium benzoate: 0.1% by mass
Fragrance: 1.0% by mass
Calcium sulfate: 0.5% by mass
Purified water: balance

### Formulation Example 3

Granules having the following formulation were prepared according to common practice.
Peucedanum japonicum extract of Production Example 3: 150.0 parts by mass
Calcium: 680.0 parts by mass
Iron: 6.8 parts by mass
Beet oligosaccharide: 1,000.0 parts by mass
Stevia extract: 10.0 parts by mass

### Formulation Example 4

Soft capsules having the following formulation were prepared according to common practice.
Peucedanum japonicum extract of Production Example 1: 30 parts by mass
Olive oil: 200 parts by mass
Glycerin fatty acid ester: 24 parts by mass
Beeswax: 24 parts by mass

### Formulation Example 5

Chocolate having the following formulation was prepared according to common practice.
Peucedanum japonicum extract of Production Example 2: 0.15 parts by mass
Chocolate: 45.0 parts by mass
Sucrose: 15.0 parts by mass
Cocoa butter: 20.0 parts by mass
Whole milk powder: 25.0 parts by mass

### Formulation Example 6

A soft drink having the following formulation was prepared according to common practice.
Peucedanum japonicum extract of Production Example 3: 0.3% by mass
Royal jelly: 1.0% by mass
Water-soluble collagen: 10.0% by mass
Coix seed extract: 1.0% by mass
Ginseng extract: 1.0% by mass
Oligosaccharide: 5.0% by mass
Sucrose: 10.0% by mass
Prune juice: 2.0% by mass
Pomegranate: 5.0% by mass
Grapefruit juice: 10.0% by mass
Grapefruit flavor: 0.7% by mass
Water: balance
Total: 100.0% by mass

This international application claims priority based on Japanese Patent Application No. 2022-040345 filed on Mar. 15, 2022, the entire contents of which are incorporated herein by reference.

1. An anti-obesity agent comprising:
an extract of a below-ground portion of Peucedanum japonicum.

2. The anti-obesity agent according to claim 1,
wherein the extract of the below-ground portion of Peucedanum japonicum is an extract obtained using a mixed solvent including water and a hydrophilic solvent.

3. The anti-obesity agent according to claim 1,
wherein the extract of the below-ground portion of Peucedanum japonicum includes, as khellactone esters, hyuganin D, Peucedanocoumarin III, cis-3′-acetyl-4′-tigloylkhellactone, trans-3′-acetyl-4′-senecioylkhellactone, isosamidin, and pteryxin.

4. The anti-obesity agent according to claim 1,
wherein the anti-obesity agent has one or more functions selected from the group consisting of a function of inhibiting cyclic AMP phosphodiesterase activity, a function of facilitating differentiation induction of preadipocytes, a function of inhibiting lipopexia, and a function of inducing cell death of adipocytes.

5. An anti-obesity composition comprising:
the anti-obesity agent according to claim 1.
